(19) United States
(12) Patent Application Publication
(10) Pub. No.: US 2025/0314596 A1
(43) Pub. Date: Oct. 9, 2025

(54) LIQUID SUBSTANCE INSPECTING APPARATUS

(71) Applicant: INTIN INC., Daegu-si (KR)

(72) Inventor: Ji Hoon KIM, Seoul (KR)

(73) Assignee: INTIN INC., Daegu-si (KR)

(21) Appl. No.: 18/961,834

(22) Filed: Nov. 27, 2024

(30) Foreign Application Priority Data
Apr. 5, 2024 (KR) 10-2024-0046798

(51) Int. Cl. G01N 21/88

(52) U.S. Cl. CPC G01N 21/8803; G01N 2201/062

(57) ABSTRACT

Disclosed is a liquid substance inspecting apparatus including: an upper housing; a battery fastened to the upper housing and supplying power; a controller; and a second terminal formed in the upper surface of the controller and coming into contact with the battery when a chamber is inserted; a Printed Circuit Board (PCB) supporter disposed below the controller, and configured to support the controller and move vertically toward the battery upon insertion of the chamber, thereby causing the second terminal to contact the battery; a lower housing formed with an insertion hole into which the chamber is inserted, in which the controller and the PCB supporter are fastened to an internal fastening structure, and which is fastened to the upper housing; and a lens unit installed inside the lower housing to enable a user or a user terminal to check a liquid substance contained in the chamber.

### CROSS-REFERENCE TO RELATED APPLICATIONS

This application claims priority to and the benefit of Korean Patent Application No. 10-2024-0046798, filed on Apr. 5, 2024, the disclosure of which is incorporated herein by reference in its entirety.

### FIELD

The present disclosure relates to a liquid substance inspecting apparatus, and more specifically, to a liquid substance inspecting apparatus that enables observation and inspection of a liquid substance.

### BACKGROUND

First, the term “liquid substance” as used herein refers to all types of bodily fluids contained in the human body, such as saliva, blood, semen, sweat, urine, and tears. More broadly, it encompasses various similar liquid substances, including both organic and inorganic compounds utilized in scientific research across fields like medicine, biology, water quality, and environmental studies.

Among liquid substances, bodily fluids are used as samples to assess a person's health or physical condition. Accordingly, people often visit specialized medical institutions to check their health based on these test results. However, visiting such institutions requires taking time out of a busy schedule and enduring inconvenient and complex procedures and diagnostic processes.

In particular, although there is often a long waiting time for a test, the actual examination or testing is typically completed quickly. In other words, undergoing an examination or test requires significant time and cost, accompanied by considerable inconvenience.

As a result, most people tolerate discomfort in their daily lives and only seek specialized medical care when experiencing unbearable pain or distress.

To address such inconveniences, personal testers have been developed, as disclosed in Korean Patent Nos. 10-2435175 and 10-2288223.

However, conventional personal testers have a high defect rate due to a large number of parts and complicated manufacturing processes. Additionally, the conventional personal testers require internal illumination for a user to visually confirm chamber insertion, resulting in a complex illumination structure.

### PRIOR ART LITERATURE

### Patent Document

(Patent Document 1) Korean Patent No. 10-2435175 (Registration Date: Aug. 18, 2022)

### SUMMARY

In view of the above, the present disclosure provides a liquid substance inspecting apparatus having a small number of parts and easy assembly of the parts, thereby reducing unit cost and defect rates.

The present disclosure also provides a liquid substance inspecting apparatus that eliminates LED lighting defects by simplifying a drive mechanism for LED illumination.

In one general aspect, there is provided a liquid substance inspecting apparatus including: an upper housing; a battery fastened to the upper housing and supplying power; a controller configured to operate by receiving power from the battery, and having an LED module mounted thereon, a first terminal formed in an upper surface of the controller and in constant contact with the battery, and a second terminal formed in the upper surface of the controller and coming into contact with the battery 120 when a chamber is inserted; a Printed Circuit Board (PCB) supporter disposed below the controller, and configured to support the controller and move vertically toward the battery upon insertion of the chamber, thereby causing the second terminal to contact the battery; a lower housing formed with an insertion hole into which the chamber is inserted, in which the controller and the PCB supporter are fastened to an internal fastening structure, and which is fastened to the upper housing; and a lens unit installed inside the lower housing to enable a user or a user terminal to check a liquid substance contained in the chamber.

The liquid substance inspecting apparatus may further include an elastic body disposed between the battery and the controller and configured to maintain a normal state when the chamber is not inserted, to contract upon the insertion of the chamber due to the vertical movement of the PCB supporter, and to return to the normal state upon removal of the chamber.

The PCB supporter may include: a contact surface two seating plates on which the controller is seated; and a contact plate connecting the two seating plates and formed at a lower position than each of the seating plates to come into contact with the chamber.

At least one surface protruding downward may be formed in a rear surface of the contact plate at a position furthest from a direction of entry of the chamber. The protruding surface of the rear surface of the contact plate may be in contact with the chamber, whereas any other part of the rear surface does not contact the chamber. The PCB supporter may move vertically by the contact between the protruding surface and the chamber.

The first terminal may be composed of a conductor, be installed near one of longitudinal ends on an upper surface of the PCB, protrude toward the battery, and have a protruding surface coming into contact with the battery and formed as a diagonal surface of a predetermined length. The second terminal may be composed of a conductor and be installed at the upper surface of the PCB so as to be spaced apart from the first terminal, and be spaced apart from the battery by a first distance when the chamber is not inserted. The first distance may be smaller than a distance according to the vertical movement.

The second terminal may be at least one protruding plate having both ends protruding diagonally.

The fastening structure of the lower housing may comprise a plurality of fastening rods formed to protrude from an inner bottom surface. At least one stepped portion may be formed to protrude laterally on the side of each of the plurality of fastening rods. The PCB supporter may be inserted to be seated on a step formed by a stepped portion, without the chamber being inserted.

A distance between the lens surface of the lens part and the PCB supporter may be shorter than the first distance.

### BRIEF DESCRIPTION OF THE DRAWINGS

FIG. 1 is a perspective view of a liquid substance inspecting apparatus according to an embodiment of the present disclosure.

FIG. 2 is an exploded perspective view of a liquid substance inspecting apparatus according to an embodiment of the present disclosure.

FIG. 3 is a perspective view of a controller in a liquid substance inspecting apparatus according to an embodiment of the present disclosure.

FIG. 4 is a front perspective view of a PCB supporter in a liquid substance inspecting apparatus according to an embodiment of the present disclosure.

FIG. 5 is a rear perspective view of a supporter in a liquid substance inspecting apparatus according to an embodiment of the present disclosure.

FIG. 6 is a drawing for explaining a process of attaching the main components to a lower housing in a liquid substance inspecting apparatus according to an embodiment of the present disclosure.

FIG. 7 is a drawing showing the arrangement between components when a chamber is not inserted into a liquid substance inspecting apparatus according to an embodiment of the present disclosure.

FIG. 8 is a drawing showing the arrangement of components while a chamber is inserted into a liquid substance inspecting apparatus according to an embodiment of the present disclosure.

FIG. 9 is a drawing showing the arrangement between each component when the chamber is fully inserted into a liquid substance inspecting apparatus according to an embodiment of the present disclosure.

### DETAILED DESCRIPTION

Description will now be given in detail according to exemplary embodiments disclosed herein, with reference to the accompanying drawings. For the sake of brief description with reference to the drawings, the same or equivalent components may be provided with the same or similar reference numbers, and description thereof will not be repeated. In addition, in the following description of the embodiments, a detailed description of known functions and configurations incorporated herein will be omitted when it may impede the understanding of the embodiments.

While terms including ordinal numbers, such as “first” and “second,” etc., may be used to describe various components, such components are not limited by the above terms. The above terms are used only to distinguish one component from another.

The singular forms are intended to include the plural forms, unless the context clearly indicates a different meaning.

In this application, the described steps may be carried out in any sequence, except in cases where a clearly defined cause-and-effect relationship necessitates a specific order.

It will be further understood that the terms “comprise”, “include”, “have”, etc. when used in this specification, specify the presence of stated features, integers, steps, operations, elements, components, and/or combinations of them but do not preclude the presence or addition of one or more other features, integers, steps, operations, elements, components, and/or combinations thereof.

Hereinafter, the present disclosure will be described with reference to the attached drawings.

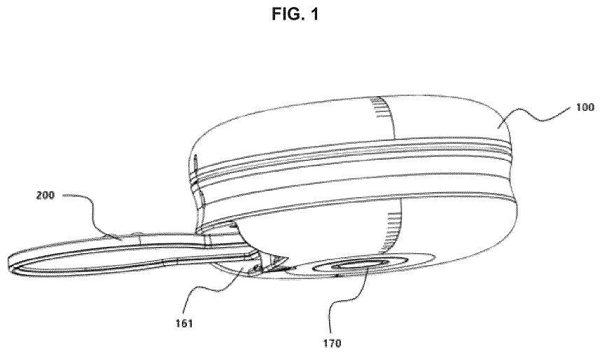

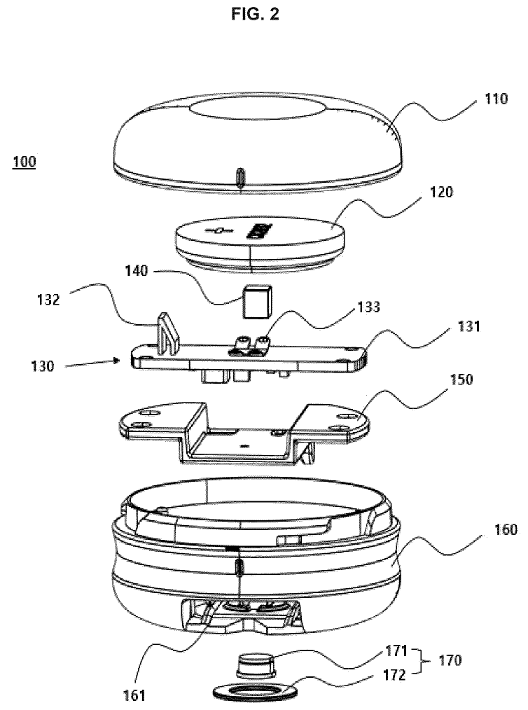

FIG. 1 is a perspective view of a liquid substance inspecting apparatus according to an embodiment of the present disclosure, and FIG. 2 is an exploded perspective view of a liquid substance inspecting apparatus according to an embodiment of the present disclosure.

Referring to FIGS. 1 and 2, a chamber 200 containing a liquid substance sample is inserted and secured through an insertion hole 161 into a liquid substance inspecting apparatus 100 according to an embodiment of the present disclosure. The liquid substance inspecting apparatus 100 allows a user to visually check (observe) the liquid substance sample contained in the chamber 200 through a lens part 170 installed on the back, or to check (observe) the liquid substance sample via a screen of a user terminal (e.g., a smartphone) located below the lens unit 170.

The liquid substance inspecting apparatus 100 according to an embodiment of the present disclosure may include an upper housing 110, a battery 120, a controller 130, an elastic body 140, a PCB supporter 150, a lower housing 160, and the lens part 170.

The upper housing 110 is fastened to the lower housing 160 and, together with the lower housing 160, seals and protects the internal components 120 to 150 and 170. The upper housing 110 functions as a lid and has a fastening structure for connecting or fastening the battery 120. The fastening structure falls within the scope of conventional technology, so a detailed description thereof is omitted.

The battery 120 functions to supply power to the controller 130 and is coupled or fastened to the upper housing 110.

The controller 130 includes various chips and components, such as an LED module, and operates by receiving power from the battery 120. When the chamber 200 is not inserted, a power line to the battery 120 is turned off, and when the chamber 200 is inserted, the power line to the battery 120 is turned on.

The PCB supporter 150 is located below the controller 130 and supports the controller 130 by contacting at least a portion of the controller 130, and moves vertically by an external force, moving the controller 130 together.

The elastic body 140 is positioned between the battery 120 and the controller 130, and is formed of an insulating material or in an insulating state to prevent a current flow between the battery 120 and the controller 130. When the chamber 200 is not inserted, the elastic body 140 maintains a state in which the elastic body 140 is not stretched or contracted, that is, does not have elasticity, thereby making a gap between the battery 120 and the controller 130 a first set gap. When the chamber 200 is inserted, the elastic body 140 is in a state in which the elastic body 140 is contracted and has elasticity. When the elastic body 140 is in a contracted state, the gap between the battery 120 and the controller 130 becomes a second set gap that is smaller than the first set gap, and the second set gap may be equal to a length of the elastic body 140 in the contracted state. The elastic body 140 pushes the controller 130 with elasticity generated when the chamber 200 is removed after the chamber 200 is inserted, thereby making the gap between the battery 120 and the controller 130 the first set gap.

For example, the elastic body 140 may be composed of a sponge, a spring covered with an insulating material, or the like. In some cases, this elastic body 140 may be omitted from the configuration of the liquid substance inspecting apparatus 100 according to an embodiment of the present disclosure.

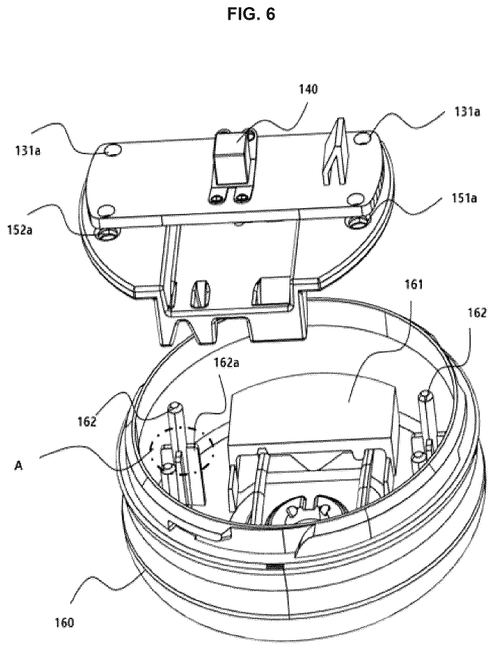

The lower housing 160 has the lens part 170 installed at a rear surface thereof, with the PCB supporter 150 installed at the lens part 170 and the controller 130 installed at the PCB supporter 150. For example, referring to FIG. 6, a plurality of fastening rods 162 protruding from an inner bottom surface of the lower housing 160 are installed at the lower housing 160, and the PCB supporter 150 and a part of the controller 130 may be fitted and fastened to each fastening rod 162.

The lens part 170 includes a lens 171 and a fastening ring 172 that secures the lens 171 to the lower housing 160. The lens part 170 enables a user or a user terminal to observe a liquid substance contained in the chamber 200, which is inserted into the liquid substance inspecting apparatus 100 through the insertion hole 161, from the outside. The lens part 170 may be installed either outside or inside the lower housing 160.

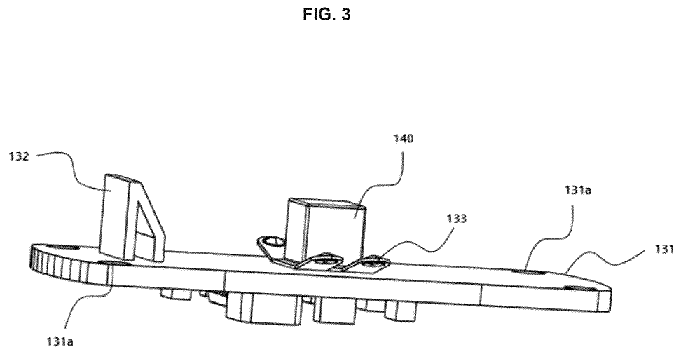

FIG. 3 is a perspective view of a controller in a liquid substance inspecting apparatus according to an embodiment of the present disclosure. Referring to FIG. 3, the controller 130 may include a Printed Circuit Board (PCB) 131, a first terminal 132, a second terminal 133, and a plurality of fastening grooves 131a.

The first terminal 132 is composed of a conductor and is installed on an upper surface (the surface close to the battery) of the PCB 131. The first terminal 132 is shaped to maintain constant contact with a battery 120. For example, if the battery 120 is a coin battery, the first terminal 132 is installed near one of the longitudinal ends of the PCB 131 and protrudes vertically from the PCB 131 by a predetermined height. The first terminal 132's surface coming into contact with the battery 120 is formed as a diagonal surface of a predetermined length, ensuring that the diagonal surface contacts a side surface of the coin battery. The predetermined height may be greater than a distance by which the PCB supporter 150 moves vertically due to an external force, that is, the chamber 200. The first terminal 132 is either a positive electrode or a negative electrode. Preferably, the first terminal 132 may be the positive electrode.

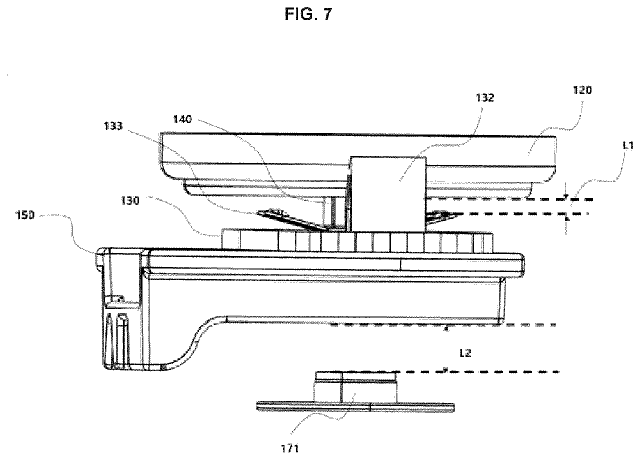

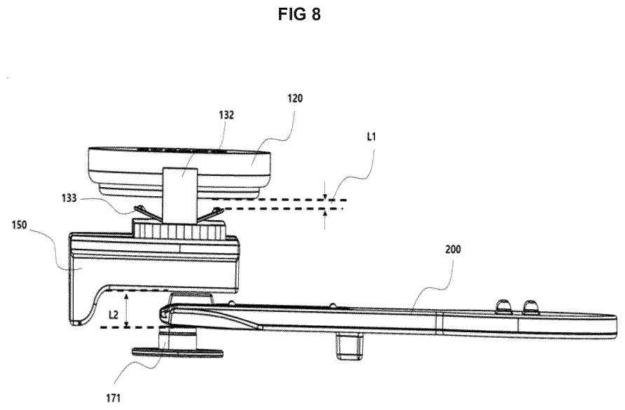

The second terminal 133 is composed of a conductor. The second terminal 133 is installed in a predetermined area on the upper surface (the surface close to the battery) of the PCB 131. The predetermined area is preferably a central portion of the upper surface of the PCB 131, but aspects of the present disclosure are not limited thereto. The second terminal 133 is configured to be spaced apart from the battery 120 by a first distance when the chamber 200 is not inserted, and to come into contact with the battery 120 when the chamber 200 is inserted. The first distance corresponds to a first gap L1 illustrated in FIGS. 7 and 8, and may be shorter than the distance the PCB supporter 150 moves vertically by the chamber 200.

For example, the second terminal 133 may be configured as at least one protruding plate with both ends protruding diagonally from the upper surface of the PCB 131. A protrusion plate refers to a terminal in the shape of a plate. As the second terminal 133 protrudes diagonally, when the controller 130 moves vertically and comes into contact with the battery 120, the end of the second terminal is lowered toward the upper surface of the PCB 131 by a force applied by the battery 120, thereby widening the contact surface with the battery 120. The second terminal 133 may be manufactured in various shapes other than a protruding plate.

The second terminal 133 may be either a positive electrode or a negative electrode. When the first terminal 132 is a positive electrode, the second terminal 133 functions as a negative electrode. This second terminal 133 functions as a switch that supplies power from the battery 120 to the controller 130 depending on whether the chamber 200 is inserted or not.

A plurality of fastening grooves 131a are formed at the longitudinal ends of the PCB 131 and are penetrated by fastening rods 162, allowing the controller 130 to be installed at the lower housing 160.

Meanwhile, the elastic body 140 may be installed on the second terminal 133 as shown in FIG. 3, but the elastic body 140 may also be installed outside the second terminal 133.

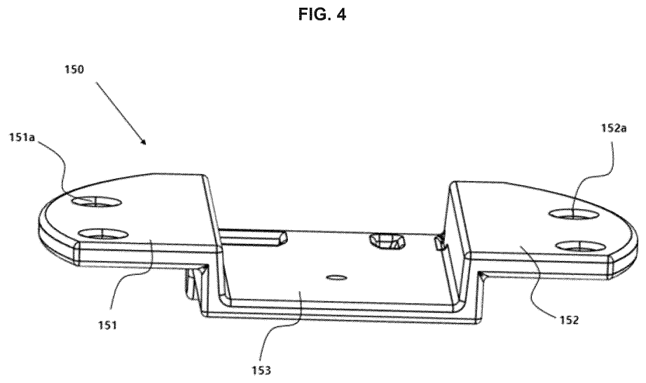

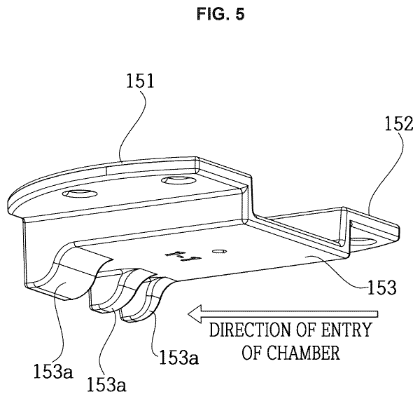

FIG. 4 is a front perspective view of a PCB supporter in a liquid substance inspecting apparatus according to an embodiment of the present disclosure, and FIG. 5 is a rear perspective view of a PCB supporter in a liquid substance inspecting apparatus according to an embodiment of the present disclosure.

Referring to FIGS. 4 and 5, a PCB supporter 150 may include two seating plates 151 and 152 on which the controller 130 is seated, and a contact plate 153 connecting the two seating plates 151 and 152 and formed at a lower position than each of the seating plates 151 and 152 to come into contact with the chamber 200. An LCD module, semiconductor chip, or element mounted on the controller 130 may be positioned in a space formed by the seating plates 151 and 152 and the contact plate 153.

In the seating plates 151 and 152, through-holes 151a and 152a may be formed at positions corresponding to the positions of the fastening grooves 131a.

The through-holes 151a and 152a, similarly to the fastening grooves 131a, may be penetrated by the fastening rods 162, thereby allowing the PCB supporter 150 to be installed at the lower housing 160.

At least one protruding surface 153a may be formed in a rear surface of the contact plate 153 at the opposite end where the chamber 200 enters, that is, at a position furthest from a direction of entry of the chamber 200. The protruding surface 153a protrudes downward to contact the chamber 200, and forms a curved slope in the direction of entry of the chamber 200, allowing the chamber 200 to smoothly move vertically upwards upon contact with the chamber 200.

As soon as the chamber 200 is inserted into the liquid substance inspecting apparatus 100, that is, before the chamber 200 is fully engaged, any vertical movement of the PCB supporter 150 causes the second terminal 133 to contact the battery 120 and the LED module of the control unit 130 light up, resulting in unnecessary power consumption. The purpose of forming the protruding surface 153a is to prevent this unnecessary power consumption.

Except for the protruding surface 153a in the rear surface of the contact plate 153, any other part of the chamber 200 is not in contact with the contact surface. That is, only the protruding surface 153a does not contact the chamber 200, so that the LED module of the controller 130 lights up when the chamber 200 is fully inserted. Of course, the protruding surface 153a may be formed at any location on the rear surface of the PCB supporter 150.

Meanwhile, the protruding surface 153a may not be formed in the rear surface of the contact plate 153. That is, the rear surface of the contact plate 153 may be manufactured flat. In this case, the entire flat rear surface of the contact plate 153 comes into contact with the chamber 200. That is, when the chamber 200 is inserted, the chamber 200 may immediately contact the rear surface of the contact plate 153 to cause the LED module of the controller 130 to light up.

FIG. 6 is a drawing for explaining a process of attaching the main components to a lower housing in a liquid substance inspecting apparatus according to an embodiment of the present disclosure. Referring to FIG. 6, a plurality of fastening rods 162 may be installed at an inner bottom surface of a lower housing 160. The number of fastening rods 162 may be the same as the number of fastening grooves 131a or through-holes 151a and 152a formed in a controller 130 or PCB supporter 150.

Each fastening rod 162 may have at least one stepped portion 162a protruding laterally on the side to form a step A. The controller 130 and the PCB supporter 150 are sequentially fastened to the fastening rods 162. When the PCB supporter 150 is fastened, the PCB supporter 150 is inserted through the fastening rod 162 because each of the through-holes 151a and 152a has a diameter greater than a diameter near the end of each of the fastening rods 162. However, since the diameter of each of the through-holes 151a and 152a is smaller than the diameter of each of the fastening rods 162, due to the step 162a, the PCB supporter 150 may be seated on the step A. That is, the step A may serve as a stopper for the insertion of the PCB supporter 150. The controller 130 that is fastened after the PCB supporter 150 is fastened to the fastening rods 162 may be fastened to the fastening rods 162 and seated on the PCB supporter 150.

FIG. 7 is a drawing showing the arrangement between components when a chamber is not inserted into a liquid substance inspecting apparatus according to an embodiment of the present disclosure. That is, FIG. 7 is a perspective view showing the inside of an upper housing 110 and a lower housing 160 when a chamber 200 is not inserted into a liquid substance inspecting apparatus 100.

Referring to FIG. 7, in the liquid substance inspecting apparatus 100 with the chamber 200 not inserted, a battery 120 and a second terminal 133 are spaced apart from each other by a first gap L1, and a contact plate 153 of a PCB supporter 150 and a lens surface of a lens 171a are spaced apart from each other by a second gap L2. In this state, the first terminal 132 is in contact with the battery 120.

Here, even if a user turns over the liquid substance inspecting apparatus 100, the first and second gaps are maintained by the step A between the elastic body 140 and each fastening rod 162.

FIG. 8 is a drawing showing the arrangement of components while a chamber is inserted into a liquid substance inspecting apparatus according to an embodiment of the present disclosure. Referring to FIG. 8, in a state in which a chamber 200 is not yet completely inserted and fastened into a liquid substance inspecting apparatus 100, the chamber 200 is advanced to be inserted without contacting a PCB supporter 150.

Accordingly, the battery 120 and the second terminal 133 are spaced apart by a first gap L1, and the contact plate 153 of the PCB supporter 150 and the lens surface of the lens 171 are spaced apart by a second gap L2. At this point, the chamber 200 is inserted between the contact plate 153 and the lens surface of the lens 171.

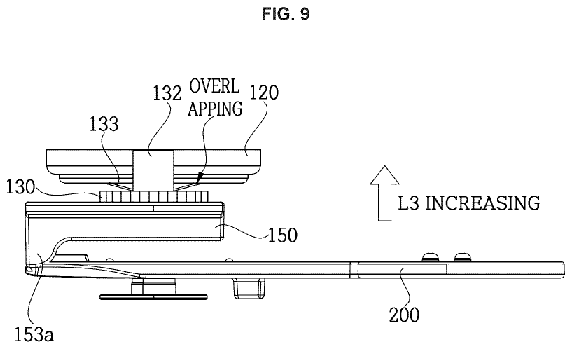

FIG. 9 is a drawing showing the arrangement between each component when the chamber is fully inserted into a liquid substance inspecting apparatus according to an embodiment of the present disclosure. Referring to FIG. 9, when a chamber 200 is fully inserted into a liquid substance inspecting apparatus 100, the chamber 200 comes into contact with a protruding surface 153a of a PCB supporter 150, thereby pushing the PCB supporter 150 vertically. That is, the chamber 200 fully inserted into the liquid substance inspecting apparatus 100 raises the PCB supporter 150 and a controller 130 by L3.

L3 is proportional to a thickness of the chamber 200 and may be greater than L1. For example, when L1 is 0.67 mm, L3 may be greater than 0.67 mm and less than 1.5 mm. When L3 is smaller than 0.67 mm, a second terminal 133 does not come into contact with the battery 120 even if the PCB supporter 150 and the controller 130 move vertically. When L3 is greater than 2 mm, the second terminal 133 overlaps the battery 120 in a larger area, which may result in damage to the second terminal 133.

As such, when the second terminal 133 comes into contact with the battery 120 through overlapping, the first terminal 132 continues to be in contact with the battery 120, so power from the battery 120 is supplied to the controller 130 and the LED module mounted on the controller 130 lights up.

According to an embodiment of the present invention, the present disclosure uses a single substrate and a drive mechanism in which the substrate moves vertically upon insertion of a chamber, thereby reducing defect rates and unit costs and eliminating LED lighting defects caused by frequent use.

The technical features disclosed in each embodiment of the present disclosure are not limited to a corresponding embodiment, and unless incompatible with each other, the technical features disclosed in each embodiment may be applied in combination to other embodiments.

Therefore, although each embodiment is described mainly about an individual technical feature, the technical features of the embodiments of the present disclosure may be applied in combination, unless incompatible with each other.

The present disclosure is not limited to the above-described embodiments and the accompanying drawings, and various modifications and changes may be made in view of a person skilled in the art to which the present disclosure pertains. Therefore, the scope of the present disclosure should be determined by the scope of the appended claims, and equivalents thereof.

What is claimed is:

1. A liquid substance inspecting apparatus comprising:
an upper housing;
a battery fastened to the upper housing and supplying power;
a controller configured to operate by receiving power from the battery, and having an LED module mounted thereon, a first terminal formed in an upper surface of the controller and in constant contact with the battery, and a second terminal formed in the upper surface of the controller and coming into contact with the battery 120 when a chamber is inserted;
a Printed Circuit Board (PCB) supporter disposed below the controller, and configured to support the controller and move vertically toward the battery upon insertion of the chamber, thereby causing the second terminal to contact the battery;
a lower housing formed with an insertion hole into which the chamber is inserted, in which the controller and the PCB supporter are fastened to an internal fastening structure, and which is fastened to the upper housing;
a lens unit installed inside the lower housing to enable a user or a user terminal to check a liquid substance contained in the chamber.

2. The liquid substance inspecting apparatus of claim 1, further comprising an elastic body disposed between the battery and the controller and configured to maintain a normal state when the chamber is not inserted, to contract upon the insertion of the chamber due to the vertical movement of the PCB supporter, and to return to the normal state upon removal of the chamber.

3. The liquid substance inspecting apparatus of claim 1, wherein the PCB supporter comprises:
a contact surface two seating plates on which the controller is seated; and
a contact plate connecting the two seating plates and formed at a lower position than each of the seating plates to come into contact with the chamber.

4. The liquid substance inspecting apparatus of claim 3, wherein:
at least one surface protruding downward is formed in a rear surface of the contact plate at a position furthest from a direction of entry of the chamber,
the protruding surface of the rear surface of the contact plate is in contact with the chamber, whereas any other part of the rear surface does not contact the chamber, and
the PCB supporter moves vertically by the contact between the protruding surface and the chamber.

5. The liquid substance inspecting apparatus of claim 1, wherein:
the first terminal is composed of a conductor, is installed near one of longitudinal ends on an upper surface of the PCB, protrudes toward the battery, and has a protruding surface coming into contact with the battery and formed as a diagonal surface of a predetermined length,
the second terminal is composed of a conductor and is installed at the upper surface of the PCB so as to be spaced apart from the first terminal, and is spaced apart from the battery by a first distance when the chamber is not inserted, and
the first distance is smaller than a distance according to the vertical movement.

6. The liquid substance inspecting apparatus of claim 5, wherein the second terminal is at least one protruding plate having both ends protruding diagonally.

7. The liquid substance inspecting apparatus of claim 1,
the fastening structure of the lower housing comprises a plurality of fastening rods formed to protrude from an inner bottom surface, and
at least one stepped portion is formed to protrude laterally on the side of each of the plurality of fastening rods, and the PCB supporter is inserted to be seated on a step formed by a stepped portion, without the chamber being inserted.
